(19) United States
(12) Patent Application Publication
(10) Pub. No.: US 2015/0100002 A1
(43) Pub. Date: Apr. 9, 2015

(54) SKIN CARE DEVICE WITH MULTIPLE FUNCTIONS

(71) Applicant: So-young CHOI, Seongnam (KR)

(72) Inventor: So-young CHOI, Seongnam (KR)

(21) Appl. No.: 14/476,949

(22) Filed: Sep. 4, 2014

(30) Foreign Application Priority Data
Oct. 7, 2013 (KR) 10-2013-0118926

(51) Int. Cl. A61N 7/00; A61N 5/06; A61M 35/00; A61N 1/20

(52) U.S. Cl. CPC A61N 7/00; A61N 1/20; A61N 5/0616; A61M 35/003; A61N 2005/0659; A61N 2005/0651; A61N 2005/0644; A61N 2005/0647; A61N 2007/0034
USPC 601/2; 607/75; 607/90; 607/3; 604/20

(57) ABSTRACT

The present invention provides a skin care device with multiple functions. The skin care device includes a main body having a first connecting terminal formed on the upper portion thereof; a plurality of skin care heads individually and detachably combined with the main body, the skin care heads each having a second connecting terminal formed on the lower portion thereof and having at least one of a galvanic current generator, an ultrasonic vibrator, or a near-infrared light emitting diode (LED) installed therein; and a product identification displaying part displays an identification number of the heads or a relevant image of the heads on a display screen when the first connecting terminal and the second connection terminal are connected each other. The skin care device allows users to care their skin effectively through a variety of beneficial functions such as galvanic electric current, ultrasonic vibrations, and near-infrared LED.

### BACKGROUND OF THE INVENTION

1. Field of the Invention

The present invention relates to a skin care device with multiple functions, more particularly to a skin care device having a plurality of skin care heads individually and detachably combined with the main body of the device, wherein if a skin care head is combined with the main body, the skin care head is instantly recognized by the device and its product identification number and image are simultaneously displayed on a display screen.

2. Description of the Related Art

Human skin starts aging in one's twenties and people's facial appearance looks old with time as well. In particular, the facial skin determines decisively another person's first impression. People guess other people's age by looking at how their skin looks. Therefore, people naturally have made efforts to look younger and more beautiful. At the same time, people have strived to delay the aging of their facial skin.

Skin aging is a natural phenomenon but its speed may differ significantly with external factors. The most representative factors include the effect of active oxygen produced on the skin, and exposure to ultraviolet rays.

Therefore, natural skin aging that comes with age cannot be controlled with human effort, but eliminating or reducing the external factors, for example, the elimination of active oxygen produced on the skin, the protection of ultraviolet rays, or the acceleration of creation of new skin cell may slow down skin aging and make the skin look younger and prettier for one's age.

Active oxygen, one of the skin aging factors, is an oxygen residue of highly oxidative that is produced inside body while inhaled oxygen is metabolized. However, it is also produced when the skin is exposed to ultraviolet rays.

Such active oxygen destroys genetic information of deoxyribonucleic acid (DNA) and collapses cell membrane by attacking normal DNA and cell groups. As a result, abnormal cell protein is created. In particular, since facial skin is more likely to be exposed to ultraviolet rays, active oxygen can be made more frequently. In this process, the fat substance inside the cell membrane of the facial skin is oxidized and thus lipid peroxide is created. When it is combined with protein, senile pigment named lipofuscin is produced, causing senile age spots. It also influences on melanin pigment, creating freckles on the facial skin. Furthermore, it spoils collagen which is skin resilience fiber, leading to sagging or wrinkled skin.

Naturally, a variety of skin care devices have been developed to tackle the problems mentioned above. Those devices include vibration massager, infrared rays treatment apparatus, ultrasonic treatment apparatus and thermotherapy device. They are independently used according to a user's purpose and are beneficial to skin care.

There is a problem, however, that users have to purchase all these products for skin care, which is financially burdensome. What's more, as those devices are quite big it is not convenient to carry or store them, making them not practical.

### BRIEF SUMMARY OF THE INVENTION

One of the objectives of the present invention is to solve the problems mentioned above. According to the present invention, a plurality of skin care heads can be individually separated from or combined with the main body of a skin care device, and when a skin care head is combined with the main body, it is immediately recognized by the device and its product identification number and image are simultaneously displayed on a display screen. This skin care device with multi-functions allows users to care their skin effectively through a variety of beneficial functions such as galvanic electric current, ultrasonic vibrations, and near-infrared LED.

The skin care device according to the present invention includes a main body of a cosmetic container shape, the body having a first connecting terminal formed on the upper portion thereof; a plurality of skin care heads individually and detachably combined with the upper portion of the main body, the heads each having a second connecting terminal formed on the lower portion thereof, the second connecting terminal electrically being connected with the first connecting terminal; and a product identification displaying part displaying an identification number of the heads or a relevant image of the product on a display screen when the first connecting terminal and the second connection terminal are connected each other.

The product identification displaying part controlled by a micro-computer is to display product information on the display screen. The micro-computer has each unique number inputted for each skin care head, and according to the each inputted number, the information of a skin care head is displayed on the display screen when the skin care head is connected with the main body.

If a skin care head is connected to the main body, the skin care head instantly starts to work, and if it is disconnected from the main body, its power is turned off.

A plurality of skin care heads are individually and detachably connected with the main body, according to a user's purpose.

In one embodiment, the plurality of skin care heads comprise a first skin care head, a second skin care head, a third skin care head, a fourth skin care head, a fifth skin care head and a sixth skin care head wherein the first skin care head has a galvanic current generator and an ultrasonic vibrator installed therein, the second skin care head, the third skin care head, the fourth skin care head, the fifth skin care head and the sixth skin care head each has a galvanic current generator, an ultrasonic vibrator and a near-infrared LED installed therein.

In another embodiment, the skin care device further comprises a negative ion sprayer configured to combine with the upper portion of the main body wherein the negative ion sprayer sprays ultrafine water of negative ion onto a user's face.

In still another embodiment, the skin care device further comprises a mask attachable to a user's face and electrically connected to the main body of the device, wherein a conductive clip corresponding to the negative pole is connected to a user's hand and another conductive clip corresponding to the positive pole is connected to the mask.

As seen above, the first connecting terminal is formed on the upper portion of the main body, the second connecting terminal is formed inside a skin care head, and a skin care head can be detachably combined with the upper portion of the main body. Thus, a plurality of skin care heads are individually and detachably connected to the main body, and when a skin care head is connected to the main body, it is instantly recognized by the main body and starts working.

Further, if one of the skin care heads is connected to the main body, a user can easily recognize which skin care head is connected because the identification number and the product image of the connected skin care head are displayed on the display screen.

Furthermore, more effective skin care can be made because galvanic current, ultrasonic vibration and near-infrared LED are transmitted onto a user's skin while the device is working

Moreover, if the negative ion sprayer is connected to the main body, the moisture level of the skin can be maintained helping a user keep hydrated skin because the negative ion sprayer sprays ultrafine water of negative ion on the user's face.

Also, if the mask is connected with the main body, the nutritional contents of cosmetics applied on the facial skin will be absorbed more promptly and evenly into the skin, resulting in higher absorption.

### BRIEF DESCRIPTION OF THE DRAWING

FIG. 1 is a perspective view of a skin care device according to the present invention, with a skin care head being separated from the main body thereof.

FIG. 2 is a front view of a skin care device according to the present invention, with a skin care head being separated from the main body thereof.

FIG. 3 a perspective view of a skin care device according to the present invention, with another skin care head being separated from the main body thereof.

FIG. 4 is a perspective view of a plurality of skin care heads according to the present invention.

FIG. 5 is a perspective view of a skin care device according to the present invention, with a negative ion sprayer being separated from the main body thereof

FIG. 6 is a configuration diagram illustrating a mask connected to the main body of a skin care device according to the present invention.

### DETAILED DESCRIPTION OF THE INVENTION

Some embodiments of the present invention will be described in detail with reference to the accompanying drawings.

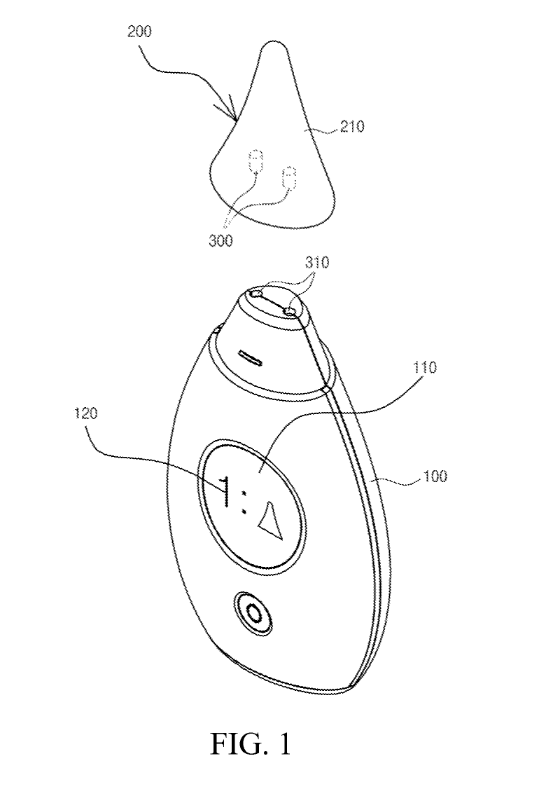

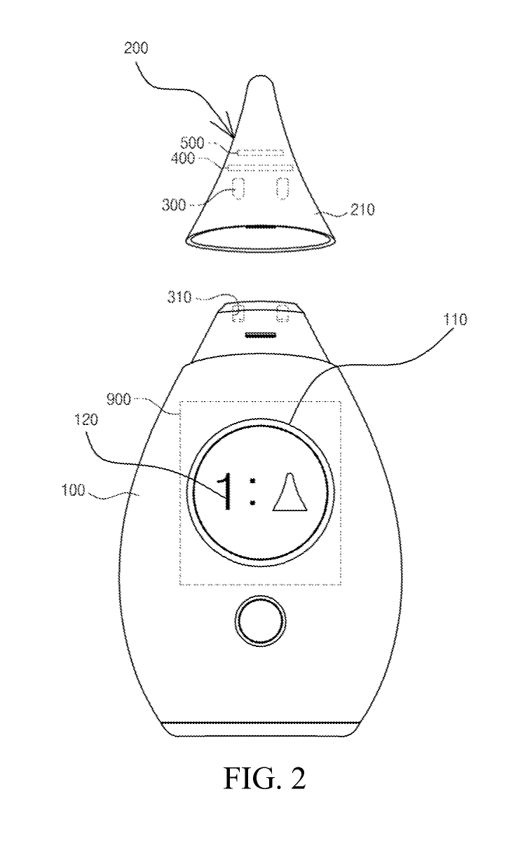

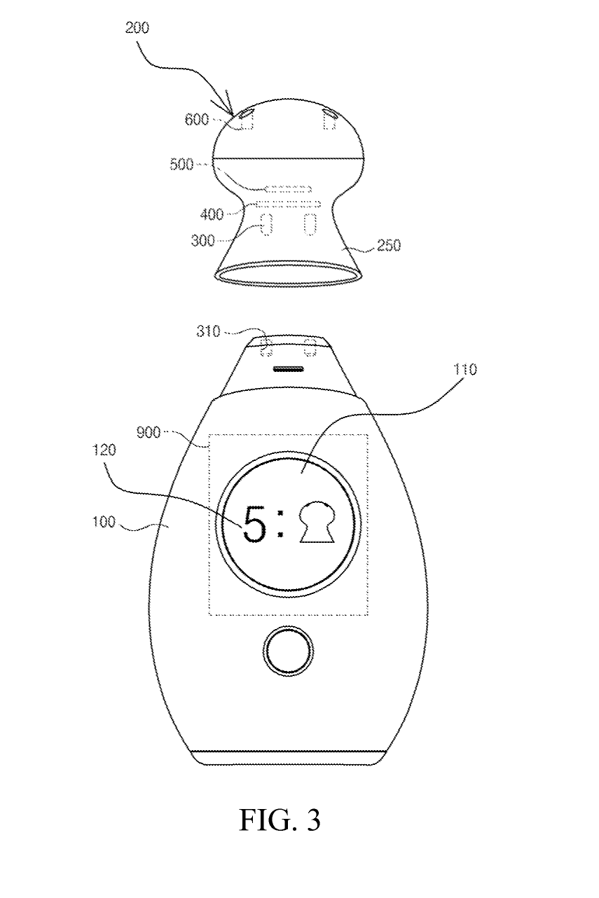

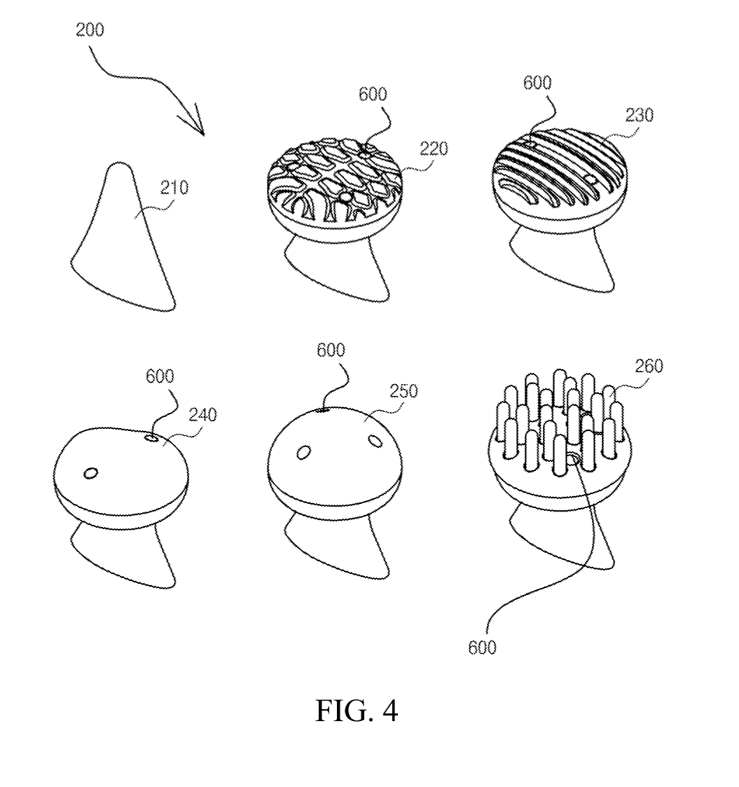

In one embodiment, as illustrated in FIGS. 1-4, a skin care device according to the present invention includes a main body 100 of a cosmetic container shape, the body having a first connecting terminal 310 formed on the upper portion thereof a plurality of skin care heads 200 individually and detachably combined with the upper portion of the main body 100, the heads 200 each having a second connecting terminal 300 formed on the lower portion thereof, the second connecting terminal 300 electrically being connected with the first connecting terminal 310; and a product identification displaying part 120 displaying an identification number of the heads or a relevant image of the heads on a display screen 110 when the first connecting terminal 310 and the second connection terminal 300 are connected each other.

The skin care device has a shape of a usual cosmetic container if the main body 100 is combined with a skin care head 200. The plurality of skin care heads 200 are individually used with one main body 100.

In one embodiment, the plurality of skin care heads 200 comprise a first skin care head 210, a second skin care head 220, a third skin care head 230, a fourth skin care head 240, a fifth skin care head 250 and a sixth skin care head 260 wherein the first skin care head 210 has a galvanic current generator 500 and an ultrasonic vibrator 400 installed therein, and wherein the second skin care head 220, the third skin care head 230, the fourth skin care head 240, the fifth skin care head 250 and the sixth skin care head 260 each have a galvanic current generator 500, an ultrasonic vibrator 400 and a near-infrared LED 600 installed therein.

The first skin care head 210, the second skin care head 220, the third skin care head 230, the fourth skin care head 240, the fifth skin care head 250 and the sixth skin care head 260 each are shaped differently. This is because each different shape may bring more effective skin care.

The first skin care head 210, the second skin care head 220, the third skin care head 230, the fourth skin care head 240, the fifth skin care head 250, the sixth skin care head 260, and the negative ion sprayer 700 have a same terminal structure such that each of them works with the first connecting terminal 310 and the second connecting terminal 300.

Galvanic current generated by the galvanic current generator 500 is direct current which flows constantly at low voltage from the positive pole to the negative pole. If it flows in a certain type of solution that contains acid and salt, chemical reaction occurs. Galvanic current is a direct current ranged from 60 to 80 V and chemically reacts if it passes through the skin containing more than 60 percent of moisture. It is also known that chemical reaction occurs if galvanic current passes through a human body's cell tissue and cell sap. Such galvanic current makes the skin more resilient, expands blood vessels accelerating blood circulation, and stimulates the skin helping to get better nutritious state.

Ultrasonic wave generated from the ultrasonic vibrator 400 is a sound wave that vibrates so quickly that human cannot hear. Its frequency is over 18,000 Hz. This ultrasonic vibration stimulates the skin, resulting in muscle strengthening, resilience increase and acceleration of nutrient provision into cellular tissues.

Near-infrared LED 600 expands blood vessels inside cellular tissues, increasing the blood flow. It also help accelerate cellular division, activate cell, and empower immune cell and has anti-inflammatory function. Thus, it has been used for various disease cure.

The product identification displaying part 120 controlled by a micro-computer 900 is to display product information on the display screen 110. The micro-computer 900 has each unique number inputted for each skin care head 200, and according to the each inputted number, the information on a skin care head 200 is displayed on the display screen 110 when the skin care head 200 is connected with the main body 100.

For example, when setting a unique product number for each skin care head, you can assign ‘1’ for the first skin care head 210, ‘2’ for the second skin care head 220, ‘3’ for the third skin care head 230, ‘4’ for the fourth skin care head 240, ‘5’ for the fifth skin care head 250 and ‘6’ for the sixth skin care head 260 and input a relevant product image for each skin care head into the micro-computer 900.

If the second skin care head 220 is combined with the main body 100, the first connecting terminal 310 and the second connecting terminal 300 are electrically connected each other. In this case, micro-computer 900 recognizes the connection of the second skin care head 220 and displays a relevant product identification and image on the display screen 110 of the product identification displaying part 120.

In other words, the skin care head 200 works immediately when the skin care head 200 is combined with the main body 100, while the skin care head is turned off when the skin care head 200 is separated from the main body 100. Furthermore, a plurality of skin care heads 200 are individually and detachably combined with one main body 100 and thus a specific skin care head may be used selectively according to a user's purpose.

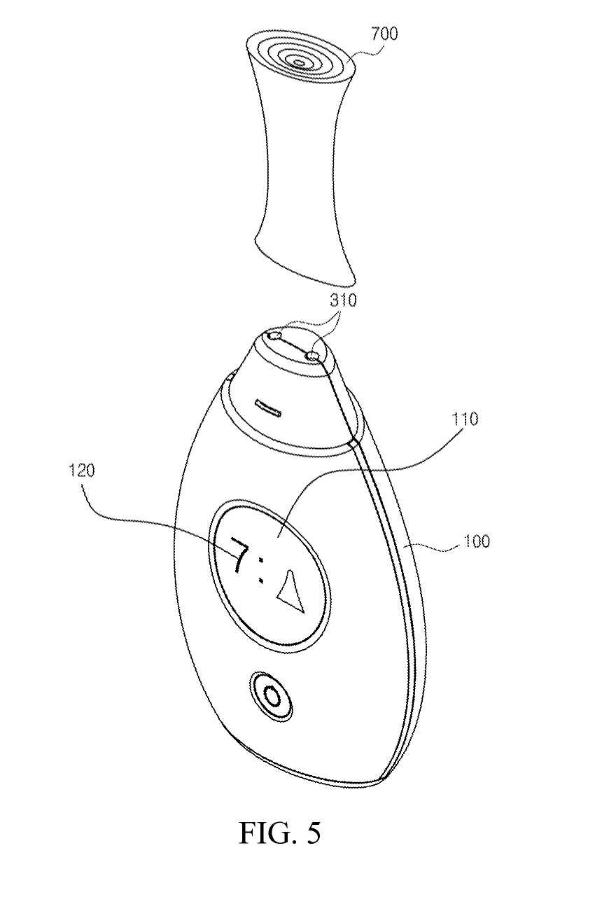

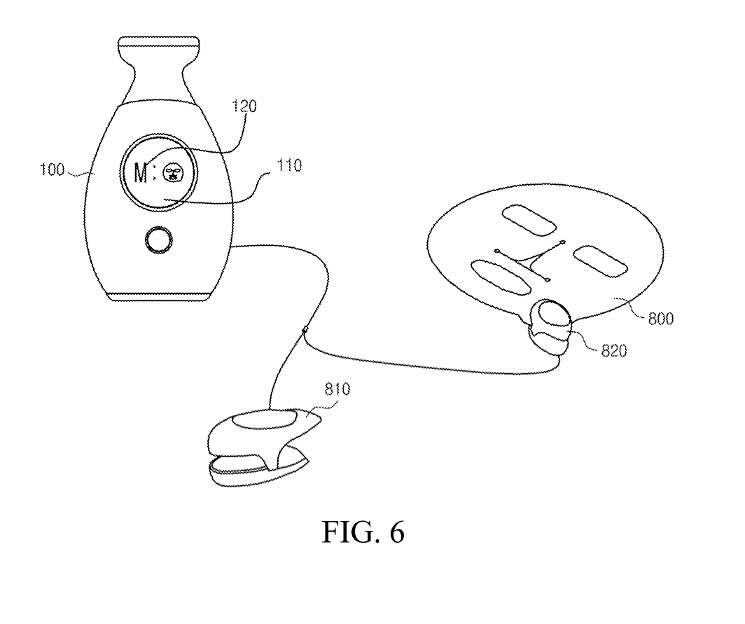

In another embodiment, as illustrated in FIG. 5, a negative ion sprayer 700 may be combined with the upper portion of the main body, which sprays ultrafine water of negative ion onto a user's face. In still another embodiment, as illustrated in FIG. 6, the skin care device includes a mask 800 attachable to a user's face and electrically connected to the main body 100 of the device. A conductive clip 810 corresponding to the negative pole is connected to a user's hand and another conductive clip 820 corresponding to the positive pole is connected to the mask 800.

Because technologies regarding skin care head, negative ion sprayer, and mask are widely used, detailed information on their specific configuration and mechanism will be omitted here.

According the present invention, one of the first skin care head 210, the second skin care head 220, the third skin care head 230, the fourth skin care head 240, the fifth skin care head 250 or the sixth skin care head 260 may be selectively connected with the main body 100. For example, the first skin care head 210 may be used for the nose area and the second and the third skin care heads 220, 230 may be used to remove skin dead cell. For a gentle massage, the fourth skin care head 240 and the fifth skin care head 250 may be used, and the sixth skin care head 260 may be used for the scalp massage.

For example, in order to care the nose area, a user may select the first skin care head 210 among the skin care heads 200. When the user combines the first skin care head with the upper portion of the main body 100, the first connecting terminal 310 and the second connecting terminal 300 are connected each other and the first skin care head 210 becomes ready to operate.

Then, micro-computer 900 recognizes that the first skin care head 210 has been connected and displays a product identification information on the display screen 110 of the product identification displaying part 120. The product identification displaying part 120 displays the number ‘1’ and the relevant image of the first skin care head. A user can now use the device on his skin, and the rest of skin care heads, which are the second skin care head 220, the third skin care head 230, the fourth skin care head 240, the fifth skin care head 250 or the sixth skin care head 260, can be selectively used after connecting with the main body 100 according to the user's purpose.

Because the skin care heads each have galvanic current generator 500 and ultrasonic vibrator 400 or galvanic current generator 500, ultrasonic vibrator 400 and near-infrared LED 600 built therein, more effective skin care can be made.

Meanwhile, if a user combines the negative ion sprayer 700 with the upper portion of the main body 100, the negative ion sprayer 700 sprays ultrafine water of negative ion onto the user's face and helps keep the skin hydrated.

When using the mask 800, after having it electrically connected with main body 100, a conductive clip corresponding to the negative pole (−) is connected to a user's hand while a conductive clip corresponding to the positive pole (+) is connected with a mask. If a user applies the mask on his face, the nutritional contents of cosmetics applied on the facial skin will be easily absorbed into the skin.

### REFERENCE NUMERALS

100: main body

110: display screen

120: product identification displaying part

200: skin care head

210: first skin care head

220: second skin care head

230: third skin care head

240: fourth skin care head

250: fifth skin care head

260: sixth skin care head

300: second connecting terminal

310: first connecting terminal

400: ultrasonic vibrator

500: galvanic current generator

600: near-infrared LED

700: negative ion sprayer

800: mask

810, 820: conductive clip

900: micro computer

What is claimed is:

1. A skin care device having multi-functions comprising:
a main body of a cosmetic container shape, the body having a first connecting terminal formed on the upper portion thereof;
a plurality of skin care heads individually and detachably combined with the upper portion of the main body, the heads each having at least one of a galvanic current generator, an ultrasonic vibrator, or a near-infrared light emitting diode (LED) installed therein, the heads each having a second connecting terminal formed on the lower portion thereof, the second connecting terminal electrically being connected with the first connecting terminal; and
a product identification displaying part displaying an identification number of the heads or a relevant image of the heads on a display screen when the first connecting terminal and the second connection terminal are connected each other.

2. The skin care device of claim 1, wherein the skin care heads comprise a first skin care head, a second skin care head, a third skin care head, a fourth skin care head, a fifth skin care head, and a sixth skin care head, the first skin care head having a galvanic current generator and an ultrasonic vibrator installed therein, the second skin care head, the third skin care head, the fourth skin care head, the fifth skin care head, and the sixth skin care head each having a galvanic current generator, an ultrasonic vibrator, and a near-infrared light emitting diode (LED) installed therein.

3. The skin care device of claim 1, wherein the device further comprises a negative ion sprayer configured to combine with the upper portion of the main body, the negative ion sprayer spraying ultrafine water of negative ion onto a user's face.

4. The skin care device of claim 1, wherein the device further comprises a mask attachable to a user's face and electrically connected to the main body of the device, a conductive clip corresponding to the negative pole being connected to a user's hand and another conductive clip corresponding to the positive pole being connected to the mask.
